(19) United States
(12) Patent Application Publication
(10) Pub. No.: US 2024/0016783 A1
(43) Pub. Date: Jan. 18, 2024

(54) RILUZOLE FOR THE TREATMENT OF ALZHEIMER'S DISEASE

(71) Applicant: Icahn School of Medicine at Mount Sinai, New York, NY (US)

(72) Inventor: Ana Pereira, New York, NY (US)

(73) Assignee: Icahn School of Medicine at Mount Sinai, New York, NY (US)

(21) Appl. No.: 18/247,561

(22) PCT Filed: Oct. 4, 2021
(86) PCT No.: PCT/US21/53403

§ 371 Date: Apr. 3, 2023

Related U.S. Application Data
(60) Provisional application No. 63/087,610, filed on Oct. 5, 2020.
(60) Provisional application No. 63/141,383, filed on Jan. 25, 2021.

(51) Int. Cl. A61K 31/428; A61P 25/28

(52) U.S. Cl. CPC A61K 31/428; A61P 25/28

(57) ABSTRACT

Disclosed herein are methods of treating Alzheimer's Disease by the administration of riluzole to patients in need thereof.

### RELATED APPLICATIONS

This application is a § 371 national stage of PCT International Application No. PCT/US2021/053403, entitled “Riluzole For The Treatment of Alzheimer's Disease” filed on Oct. 4, 2021, which claims priority to U.S. Provisional Application Nos. 63/087,610, filed Oct. 5, 2020, and 63/141,383, filed Jan. 25, 2021, which are incorporated herein by reference in their entirety.

### BACKGROUND

Alzheimer's disease (“AD”) is the most common cause of dementia, an affliction that ultimately occurs in over 43 million people worldwide. The majority of dementia cases occur after age 65, which impose an increasing burden on societies with aging populations. AD is defined biologically by the presence of a specific neuropathology of the brain: extracellular deposition of amyloid-β (Aβ) in the form of diffuse and neuritic plaques and the presence of neuropil threads within dystrophic neurites that contain aggregated, hyperphosphorylated tau protein and intraneuronal neurofibrillary tangles.

The leading model of AD pathogenesis posits that cleavage of β-amyloid precursor protein (APP) leads to the deposition of Aβ, deposition of hyperphosphorylated tau, the generation of neurofibrillary tangles (NFT), neuronal and synaptic loss, immune activation, and cognitive decline (Long and Holtzman, Cell 179, 316-17 (2019)). Thus, there are numerous potential targets for treating AD, including, for example, APP, Aβ, ApoE, neuroimmune activation, and the various biological cascades associated with each. Studies have shown, however, that the cognitive impairment associated with AD appears long after the disease begins to ravage the brain of AD patients, as evidenced by the presence of Aβ and tau deposition and of innate immune activation long before cognitive decline. And this unique pathophysiology complicates efforts to identify AD treatments that are both effective and safe.

Nevertheless, technological advances make it possible to correlate biological markers with clinical manifestations of AD and to enable disease staging and identification of potential treatments. Biological markers that can be used to diagnose and stage AD include autopsy analyses, cerebrospinal fluid (CSF) testing, in vivo proton magnetic resonance and positron emission tomography (PET) imaging of biomarkers for cerebral Aβ and tau deposition in certain areas of the brain and blood biomarkers of Aβ, total tau, phospho-tau and neurofilament, a marker of neurodegeneration. Clinical manifestations of AD can appear as impairment in learning and memory, followed by later impairments in complex attention, executive function, praxis, language, gnosis, and visuospatial function. Other manifestations of AD involve impairment in executive function or behavioral dysfunction, such as apathy or delusions. The severity of clinical dementia can be graded by use of standardized instruments, such as the Clinical Dementia Rating (CDR), which grades severity based on a composite measure of dysfunction in the areas of: memory, judgement and problem solving, orientation, involvement in community affairs, self-care, and function in home or other areas. It can also be assessed using other neuropsychological testings (Alzheimer's Disease Assessment Scale-Cognitive Subscale—ADAS-cog, ADCS, Activities of Daily Living—ADL Inventory, Neuropsychiatry Inventory—NPI) and measures of memory, executive, visuospatial, attention and language function (such as MMSE and MOCA) and others.

Despite the heavy burden on society and on aging populations, there are only four medications currently approved by the FDA for treating AD, and they are approved only for managing the cognitive impairment that are present in symptomatic AD. The drugs are: donepezil, rivastigmine, galantamine (all cholinesterase inhibitors), and memantine (an NMDA modulator). But none shows any efficacy in slowing cognitive decline or improving global functioning.

The paucity of effective AD treatments is not due to a lack of effort by investigators. Literature in the field is littered with reports of drugs that originally showed promise in animal models of AD but ultimately failed in human trials. As of this date, more than 20 compounds directed to various potential AD mechanisms provided results in animal models that were promising enough to enter phase 3 trials, but all failed at that stage. (Long and Holtzman (2019)). Just a few examples are: bapenizumab—an anti-amyloid antibody that reversed behavioral deficits in animal models; (Salloway et al., N Engl J Med. 370(4): 322-33 (2014); LMTM—a drug targeting tau that displayed evidence of AP clearance in mouse models and improved spatial learning and brain metabolism in rats (Wilcock et al., 2018); CNP520—a BACE inhibitor that reduced AP levels in rats and dogs and AP plaque deposition in a mouse AD model (Neumann et al., EMBO Mol Med. 10(11) (2018); and intravenous immunoglobulin (IVIG)—a therapy targeting the neuro-inflammatory response that showed protection from memory deficit and AP pathology in a mouse model of AD (St-Amour et al., Neuroinflammation 11:54 (2014)). All failed in phase 3 clinical trials to show efficacy and safety in humans.

One possible approach for treating AD would be to focus on neuronal populations that are susceptible to AD associated pathobiology. For example, glutamatergic dysregulation is implicated in the pathophysiology of AD, as suggested by several mechanisms. The hippocampal and neocortical atrophy visible in AD brains demonstrates degeneration predominantly in large glutamatergic pyramidal neurons (Hof et al., 1990; Hof and Morrison, 1990; Morrison and Hof, 2002a), pointing to excitatory neurons as the most vulnerable to neurodegeneration. Glutamate-mediated toxicity has been implicated as a potential mechanism of neuronal loss in AD (Hardingham and B ading, 2010). Furthermore, the neuropathophysiological hallmarks of AD, amyloid-β (Aβ) plaques and neurofibrillary tangles (NFT) formed of hyperphosphorylated tau, have also been implicated in glutamatergic dysfunction. Nevertheless, there are currently no disease-modifying therapies in AD.

In view of the widespread incidence of AD in the population, the numerous failures in finding effective AD drugs, and the absence of disease-modifying drugs, there is a need for therapies that can prevent or slow the rate of cognitive decline or can improve global functioning among patients with AD.

### SUMMARY

The present disclosure meets this need by providing for the first time a drug that can be used to slow and prevent cognitive decline associated with AD progression and to improve global functioning in AD patients.

In one embodiment, the present disclosure relates to a method of treating AD by administering to a subject in need thereof a therapeutically effective amount of riluzole for treating AD.

In one embodiment, the present disclosure relates to a method of treating AD by administering to a subject a dose of about 50 to about 300 mg per day of riluzole.

In one aspect, the present disclosure relates to a method of treating AD by administering to a subject in need thereof a dose of 100 mg per day of riluzole.

In one aspect, the present disclosure relates to a method of treating AD by administering to a subject in need thereof a dose of 50 mg of riluzole, administered twice a day.

In one embodiment, the present disclosure relates to a method of treating mild cognitive impairment (MCI) by administering to a subject in need thereof a therapeutically effective amount of riluzole for treating MCI.

In one embodiment, the present disclosure relates to a method of treating MCI by administering to a subject a dose of about 50 to about 300 mg per day of riluzole.

In one aspect, the present disclosure relates to a method of treating MCI by administering to a subject in need thereof a dose of 100 mg per day of riluzole.

In one aspect, the present disclosure relates to a method of treating MCI by administering to a subject in need thereof a dose of 50 mg of riluzole, administered twice a day.

In one embodiment, the present disclosure relates to a method of treating mild to moderate AD by administering to a subject in need thereof a therapeutically effective amount of riluzole for treating Alzheimer's disease.

In one aspect, the present disclosure relates to a method of treating mild to moderate AD by administering to a subject in need thereof a dose of about 50 to about 300 mg per day of riluzole.

In one aspect, the present disclosure relates to a method of treating mild to moderate AD by administering to a subject in need thereof a dose of 100 mg per day of riluzole.

In one aspect, the present disclosure relates to a method of treating mild to moderate AD by administering to a subject in need thereof a dose of 50 mg of riluzole, administered twice per day.

In one embodiment, the present disclosure relates to a method of treating mild AD by administering to a subject in need thereof a therapeutically effective amount of riluzole for treating Alzheimer's disease.

In one aspect, the present disclosure relates to a method of treating mild AD by administering to a subject in need thereof a dose of about 50 to about 300 mg per day of riluzole.

In one aspect, the present disclosure relates to a method of treating mild AD by administering to a subject in need thereof a dose of 100 mg per day of riluzole.

In one aspect, the present disclosure relates to a method of treating mild AD by administering to a subject in need thereof a dose of 50 mg of riluzole, administered twice per day.

In one embodiment, the present disclosure relates to a method of preventing or slowing cognitive decline, as measured using standard AD clinical tests, in a patient with mild or moderate AD by administering a dose of about 50 mg to about 300 mg per day of riluzole.

In one aspect, the present disclosure relates to a method of preventing or slowing cognitive decline, as measured using standard AD clinical tests, in a patient with mild or moderate AD by administering a dose of about 100 mg per day of riluzole.

In one aspect, the present disclosure relates to a method of preventing or slowing cognitive decline, as measured using standard AD clinical tests, in a patient with mild or moderate AD by administering a dose of about 50 mg of riluzole, administered twice per day.

In one embodiment, the present disclosure relates to a method of treating mild to moderate AD by administering to a subject in need thereof a dose of about 50 to about 300 mg per day of riluzole, wherein the subject is an apolipoprotein 4 (ApoE4) carrier.

In one aspect, the present disclosure relates to a method of treating mild to moderate AD by administering to a subject in need thereof a dose of about 100 mg per day of riluzole, wherein the subject is an apolipoprotein 4 (ApoE4) carrier.

In one aspect, the present disclosure relates to a method of treating mild to moderate AD by administering to a subject in need thereof a dose of 50 mg of riluzole, administered twice per day, wherein the subject is an apolipoprotein 4 (ApoE4) carrier.

### BRIEF DESCRIPTION OF THE DRAWINGS

FIG. 1 shows brain regions of interest for fluorodeoxyglucose-positron emission tomography (FDG-PET) analysis (top) and FDG-PET progression classifier (bottom) of glucose metabolism used for comparison between riluzole and control-AD treated groups.

FIG. 2 shows the flowchart enrollment, randomization and completion for patients in the clinical trial.

FIGS. 3A, 3B, 3C, and 3D show a comparison of the changes in posterior cingulate cerebral glucose metabolism between AD patients who received riluzole and those in the control group that received a placebo.

FIGS. 4A and 4B show the results of FDG PET imaging in patients receiving riluzole and those in the control group, who received a placebo in several brain regions of interest affected by AD.

FIG. 5A shows changes in AD progression classifier score measured through FDG-PET and 5B shows correlations between FDG PET and certain cognitive measures across patients before and after treatment.

FIGS. 6A, 6B, 6C, and 6D show a comparison of FDG PET and certain cognitive measures across patients.

### DETAILED DESCRIPTION

Riluzole and Its Clinical Applications

Riluzole is a benzothiazole derivative of the following structure:

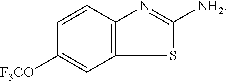 [SMILES: Nc1nc2ccc(OC(F)(F)F)cc2s1]

The chemical names for riluzole include 2-Amino-6-(trifluoromethoxy)benzothiazole and 6-(trifluoromethoxy)benzo[d]thiazol-2-amine. Riluzole has a molecular formula of C₈H₅F₃N₂OS, a molecular weight of 234.20, and a CAS number of 1744-22-5. Riluzole is soluble in dimethylformamide, dimethylsulfoxide and methanol, freely soluble in dichloromethane, sparingly soluble in 0.1 N HCl and very slightly soluble in water and in 0.1 N NaOH.

Riluzole is a glutamate modulator indicated for the treatment of amyotrophic lateral sclerosis (ALS). Riluzole is available as RILUTEK™, a film-coated tablet for oral administration containing 50 mg of riluzole, and as TIGLUTIK™, an oral suspension containing 50 mg of riluzole per 10 mL of suspension.

Studies have examined the potential of riluzole to address certain pathologies associated with AD, but only in animal models. For example, research in rodents showed that riluzole can prevent age-related cognitive decline in rodents through clustering of dendritic spines (Pereira et al., 2014b), which strengthens neural communication (Govindarajan et al., 2006; Larkum and Nevian, 2008). Additional research revealed that riluzole rescues gene expression profiles related to aging and AD in rodent models and that the most affected pathways were related to neurotransmission and neuroplasticity (Pereira et al., 2016). More recent research showed that riluzole prevented hippocampal-dependent spatial memory decline in an early-onset aggressive mouse model of AD (SXFAD) and reversed many of the gene expression changes in immune pathways (Okamoto et al., 2018). Specifically, these reversals involved microglia-related genes (Okamoto et al., 2018) thought to be critical mediators of AD pathophysiology (Streit, 2004; Butovsky et al., 2014; Colonna and Wang, 2016), including a recently identified unique population of disease-associated microglia (DAM) (Keren-Shaul et al., 2017). Nevertheless, no studies conducted to date show that riluzole is effective in treating AD in humans.

Moreover, despite its approval for treating ALS, extensive research for its use in treating neuropsychiatric and other disorders, and its commercially availability for over 20 years, riluzole is approved only for the treatment of ALS. This is due to an absence of evidence showing efficacy and safety for treating any other disease.

The present disclosure provides for the first time an AD treatment that is effective for preventing or delaying decline in cerebral glucose metabolism measured through FDA approved biomarker FDG-PET which significantly correlates and predicted cognitive function in mild to moderate AD. This is achieved through the administration of a therapeutically effective amount of riluzole to a subject in need thereof. In some embodiments, the therapeutically effective amount for treating Alzheimer's disease is a dose of about 50 mg to 300 mg per day of riluzole. In some embodiments, the therapeutically effective amount for treating Alzheimer's disease is a full dose of 100 mg that is administered as a 50 mg dose of riluzole twice per day.

The compositions of riluzole may be administered at least once per day. In some embodiments, the composition comprising riluzole is administered at least once per day. In some embodiments, riluzole is administered at least twice per day. In some embodiments, riluzole is administered one, two, three, four, or five times a day.

In the following description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the scope of the present subject matter. Aspects of the present disclosure, including the Figures, can be arranged, substituted, combined, separated, and designed in a wide variety of different configurations, all of which are contemplated herein.

References in the specification to “one embodiment”, “an embodiment”, “an example embodiment”, or “some embodiments,” etc. indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be effected in connection with other embodiments whether or not explicitly described.

The treatment of the diseases and disorders as described herein comprise the administration of any one of the formulations described herein to a subject in need thereof. Identifying the subject in need of such treatment can be in the judgment of the subject or a health care professional and can be subjective (e.g., opinion) or objective (e.g., measurable by a test or diagnostic method). Such treatment will be suitably administered to subjects, particularly humans, suffering from the disease or disorder.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### Definitions

Unless defined otherwise, all technical and scientific terms used herein have the meaning commonly understood by a person skilled in the art to which this disclosure belongs. The meaning and scope of the terms should be clear, however, in the event of any latent ambiguity, definitions provided herein take precedent over any dictionary or extrinsic definition.

As used herein, the following terms have the meanings ascribed to them below, unless specified otherwise. The terminology used herein is for the purpose of describing particular embodiments only and is not intended to be limiting of the disclosure. As used herein, the singular forms “a”, “an” and “the” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “effective amount” or “therapeutically effective amount” refers to a quantity of riluzole sufficient to achieve a desired effect or a desired therapeutic effect. In the context of therapeutic applications, the amount of riluzole administered to the subject can depend on the type and severity of the disease or symptom and on the characteristics of the individual, such as general health, age, sex, body weight and tolerance to drugs. The skilled artisan will be able to determine appropriate dosages depending on these and other factors.

As used herein, the term “treatment” includes any treatment of a condition or disease in a subject, or particularly a human, and may include: (i) preventing the disease or condition from occurring in the subject which may be predisposed to the disease but has not yet been diagnosed as having it; (ii) inhibiting the disease or condition, i.e., arresting or slowing down its progression; relieving the disease or condition, i.e., causing regression of the condition; or (iii) ameliorating or relieving the conditions caused by the disease, i.e., symptoms of the disease. “Treatment,” as used herein, could be used in combination with other standard therapies or alone.

As used herein, the methods used to measure the “cognitive decline” in AD patients refers to the standard clinical measurements used to determine the cognitive state of a subject with Alzheimer's Disease, including, for example, MMSE (mini mental state exam), MOCA (Montreal Cognitive Assessment), ADAS-Cog (Alzheimer's disease Assessment Cognitive Subscale), ADL (Activities of Daily Living), NPI (Neuropsychiatry Inventory), CDR (Clinical Dementia Rating), Logical Memory and other measures of memory, Trail Making B and others.

As used herein, the term “mild Alzheimer's Disease” or “mild AD” or “mild cognitive impairment” refers to Alzheimer's Disease patients with an MMSE (mini mental state exam) score between 19 and 27 or patients with mild cognitive impairment with clinically confirmed memory loss, as a precursor of Alzheimer's disease.

As used herein, the term “moderate or severe Alzheimer's Disease” or “moderate or severe AD” refers to MMSE lower than 19.

As used herein, the term “mild cognitive impairment” refers to mild cognitive impairment of a degenerative nature (insidious onset and gradual progression), including, for example, memory complaints, objective lower performance on a task of declarative memory. Amnestic MCI is usually a precursor of Alzheimer's disease (AD) (in AD functions/activities of daily living are impaired).

### EXAMPLES

The following examples are put forth to provide those of ordinary skill in the art with a complete disclosure and description of how to make and use the compositions, and assay, screening, and therapeutic methods of the invention, and are not intended to limit the scope of what the inventors regard as their invention.

### Example 1

The present disclosure provides for the first time that 6 months of riluzole treatment is associated with a slower decline of fluorodeoxyglucose (FDG)-positron emission tomography (FDG PET) measures of cerebral glucose metabolism compared to placebo in a double-blind, randomized, placebo-controlled trial of riluzole 50 mg twice daily in Alzheimer's disease patients. The effect was most robust (i.e. the decline was slowest) in posterior cingulate, but the effect was also observed in precuneus, lateral temporal cortex, right hippocampus and frontal cortex. The inventors discovered a significant correlation between cognitive measures and cerebral metabolism in FDG PET, which associated cerebral glucose meabolism with cognition, a key measure of brain function and performance in AD. This disclosure provides the first in-human data showing a therapeutic benefit of riluzole in patients with Alzheimer's disease.

Patient Population

Patients with a clinical diagnosis of probable Alzheimer's disease based upon neurological and neuropsychological evaluation (National Institute on Aging—Alzheimer's disease Association, NINCDS-ADRDA criteria) (McKhann et al., 1984; McKhann et al., 2011), Mini Mental State Examination (MMSE) score of 19 to 27, and 50 to 95 years were enrolled in this pilot phase 2 double-blind, randomized, placebo-controlled study. For inclusion, FDG PET baseline scans were also evaluated to confirm a lack of a frontotemporal dementia or Lewy body disease pattern of hypometabolism.

All subjects were stable on acetylcholinesterase (AChE) inhibitors for at least 2 months before starting the trial and continued to take AChE throughout the study with the exception of one subject who had never been on AChE therapy. The study was conducted at two sites (Rockefeller University Hospital and Icahn School of Medicine at Mount Sinai, both in New York City), with the approval of the Institutional Review Boards (IRB) of both Institutions. All neuroimaging was performed at Citigroup Biomedical Imaging at Weill Cornell Medicine under an IRB protocol separately approved by that Institution. Memantine was not allowed for 6-weeks prior to study entrance nor during the study duration (memantine also acts on the glutamatergic system through a different mechanism of action than riluzole). Other exclusion criteria were: abnormal liver function test (>2 times the upper limit of normal for alanine aminotransferase (ALT) or aspartate aminotransferase (AST); or bilirubin >1.5 times the upper limit of normal, positive Hepatitis Serology (Hep. B antigen+ or Hep. C antibody+), uncontrolled diabetes mellitus (Hbalc >7), chronically uncontrolled hypertension, MRI contraindication, history of brain disease, current smoker or user of nicotine-containing products, currently taking medications with evidence of glutamatergic activity or effects on brain glutamate levels such as lamotrigine, lithium, opiates, psychostimulants such as amphetamines and methylphenidate, tricyclic antidepressants, benzodiazepines and any other drug that the investigators judged might interfere with the study and others.

Randomization and Blinding

Participants were randomly assigned in a double-blind fashion to receive riluzole at a dose of 50 mg twice a day or placebo for 6 months, with age-matched cohorts of 50-74 and 75-95 years old.

The random code was generated by the hospital pharmacy, prior to study initiation, using fixed seed numbers and validated randomization software. The randomization numbers were used in sequence. In each of 2 groups, 50-74 and 75-95 years old, 24 subject numbers were randomized into balanced blocks of either 2 or 4, which were randomly assigned. Written informed consent was obtained from participants or their legally authorized representative before initiation of study procedures. Data were periodically reviewed by the study Data Safety and Monitoring Board (DSMB). Two participants had a delay in endpoint due to COVID-19 pandemic (see statistical analysis).

Study capsule dosage forms (active and placebo) were prepared by pharmacy staff in a blinded manner using over encapsulation, and opaque (size 3 capsule shells with Lactose NF used as an excipient at Rockefeller University Hospital site and 0 capsule shells with microcrystalline cellulose used as an excipient at Mount Sinai Hospital site). The active drug product contained FDA approved riluzole 50 mg tablets. For ease of use and compliance, the pharmacy packaged the blinded capsules into medication bottles or organizer trays. Bottles or Trays were labeled in a blinded manner, and included patient name, visit, and per protocol dosing instructions. Returned trays/bottles were collected by the pharmacy and patient returns, including capsule counts, were recorded by the pharmacy. All encapsulation, packaging, and labeling procedures were double verified by pharmacy staff prior to dispensing.

Procedures

All study personnel had appropriate training on study procedures and assessments. A board-certified neurologist made a neurological assessment and administered the MMSE to all subjects. FDG PET scans were acquired at baseline and at the 6-month endpoint. A neuropsychological testing battery was performed by a licensed neuropsychologist at Rockefeller University and supervised by one at Mount Sinai site at baseline, at 3 months, and at 6 months. Patients were seen once a month in clinic for clinical assessment, and blood samples were obtained at every visit for safety laboratory exams. Blood test results were evaluated by a physician not directly involved in the study in order to maintain physician-investigators blind.

Outcome Measures

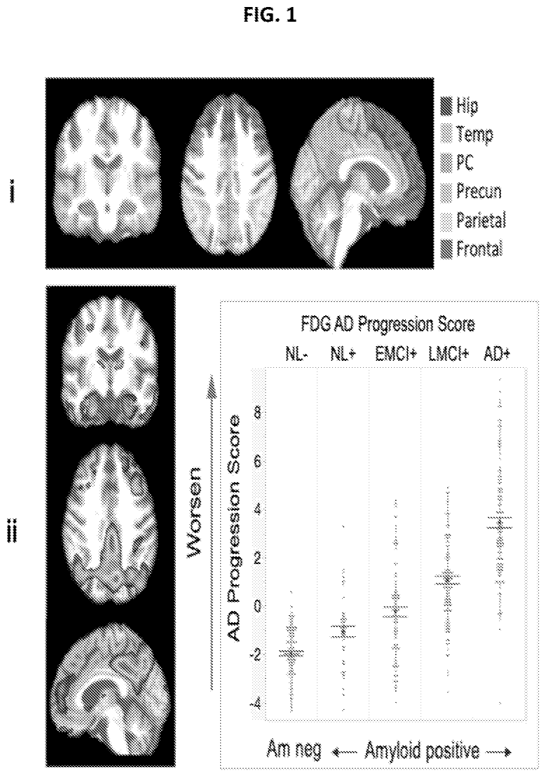

The main primary endpoint was (1) change from baseline to 6 months in cerebral glucose metabolism measured with FDG PET in posterior cingulate cortex, hippocampus, precuneus, and medial temporal, lateral temporal, inferior parietal, and frontal lobes, referred to collectively as the pre-specified regions of interest. The secondary outcome measures were neuropsychological testing (including Alzheimer's Disease Assessment Scale-Cognitive Subscale—ADAScog (Rosen et al., 1984; Mohs et al., 1997), ADCS Activities of Daily Living—ADL Inventory (Galasko et al., 1997), Neuropsychiatry Inventory—NPI (Cummings et al., 1994) total and other measures of memory, executive, visuospatial, attention and language functions for correlation with neuroimaging biomarkers as the study was not powered for a significant neuropsychological effect. Each FDG PET image was also analyzed using a previously developed AD Progression Classifier (FIG. 1A[ii]) that quantifies the degree to which a pattern of hypometabolism and preservation relative to whole brain is expressed. Increases in classifier score correspond to increased expression of a pattern of hypometabolism that corresponds to the progression of AD as validated using over 500 ADNI subjects.

FDG PET was chosen as the main primary outcome measure in this study because it is a well-established biomarker of neuronal function in AD with a clear pattern of hypometabolic and preserved brain regions (Alexander et al., 2002; Mosconi et al., 2008). Moreover, progressive hypometabolism on FDG PET strongly correlates with clinical progression in AD (Alexander et al., 2002; Landau et al., 2011; Khosravi et al., 2019).

### FIGURES

FIG. 1. A: [i] Pre-specified regions of interest, which were masked with each subject's gray tissue segment, in addition to a region defined to include the same tissue as the MRI PC region, [ii] AD progression classifier pattern, in which increasing progression scores reflect increasing expression of the pattern (subset shown) of hypometabolism (blue) and preservation (red) relative to whole brain. The progression scores of 517 test subjects from amyloid negative cognitively normal status through amyloid positive Early MCI (EMCI), late MCI (LMCI) and Alzheimer's dementia (AD) are shown, with mean and standard error, illustrating the correspondence between increased score and worsening clinical severity (data derived using FDG PET scans from ADNI, www.adni-info.org, as described in Matthews et al, 2016).

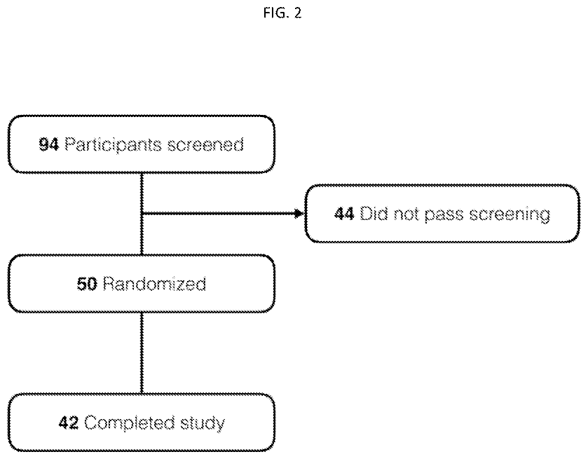

FIG. 2: Diagrams: the stages of enrollment, radomization, and trial completion.

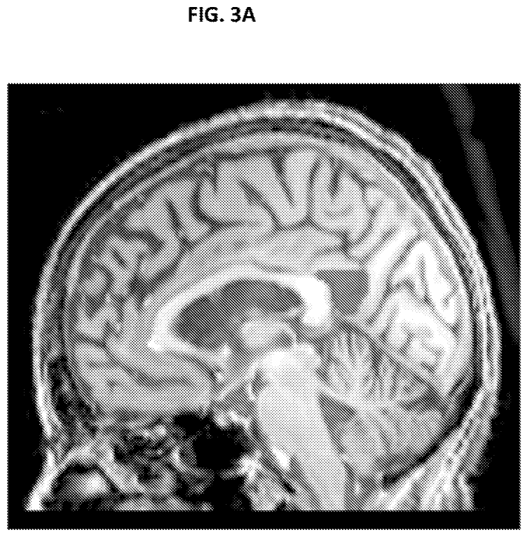

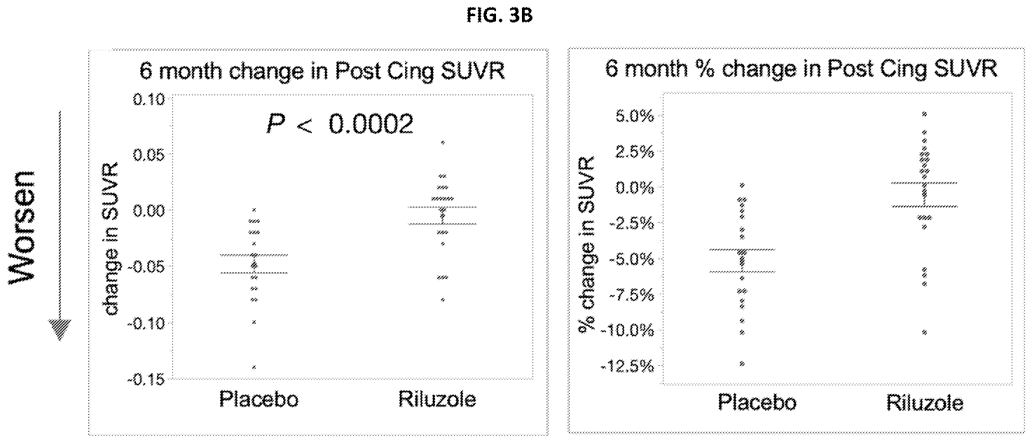

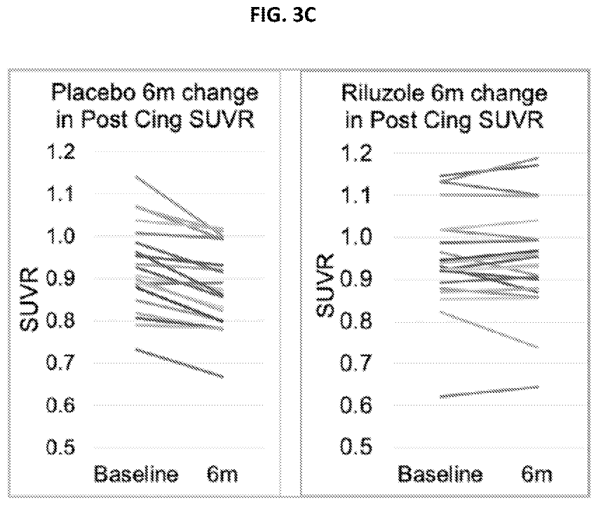

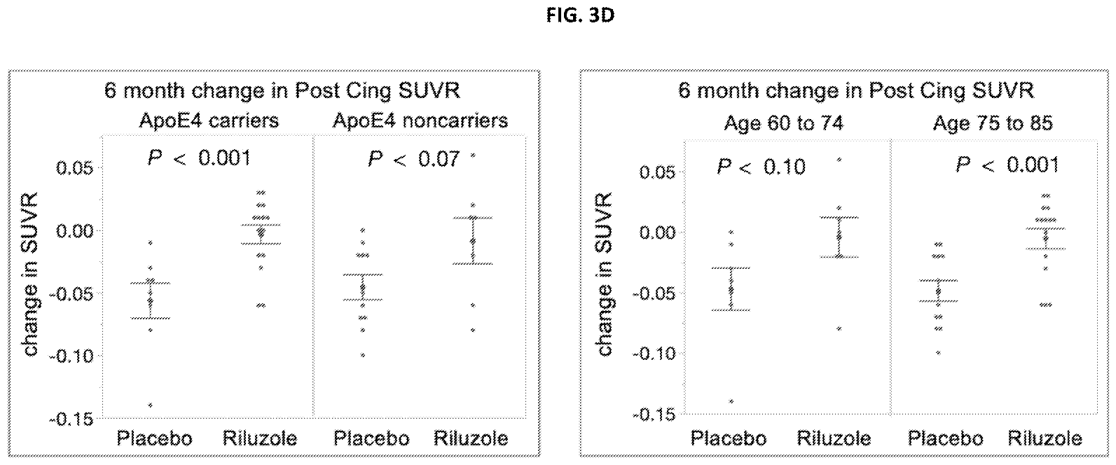

FIG. 3. (A) Posterior cingulate (PC) region of interest (representative sagittal slice) in FDG PET; (B) comparison between placebo and riluzole treated arms of the absolute and percentage change in PC FDG SUVR over the 6-month treatment period; (C) individual change from baseline to follow up in PC SUVR in placebo (left) and riluzole (right) treated arms; and (D) comparison of change in PC SUVR by ApoE4 carrier and non-carrier subgroups, and by younger and older age groups.

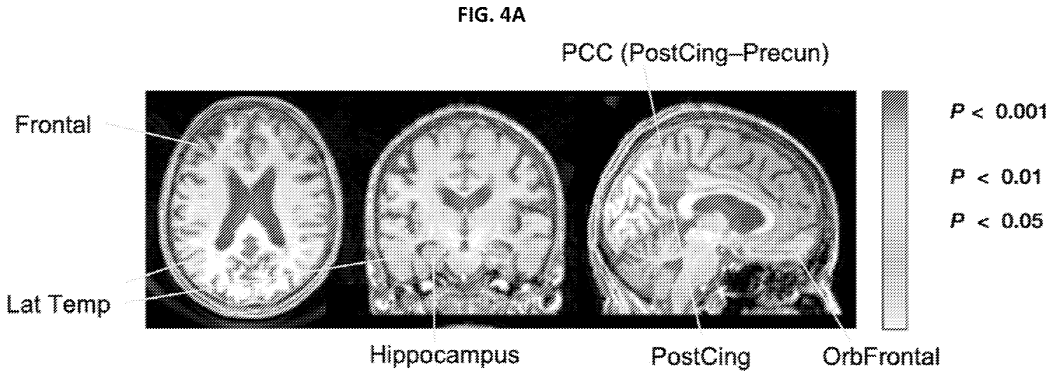

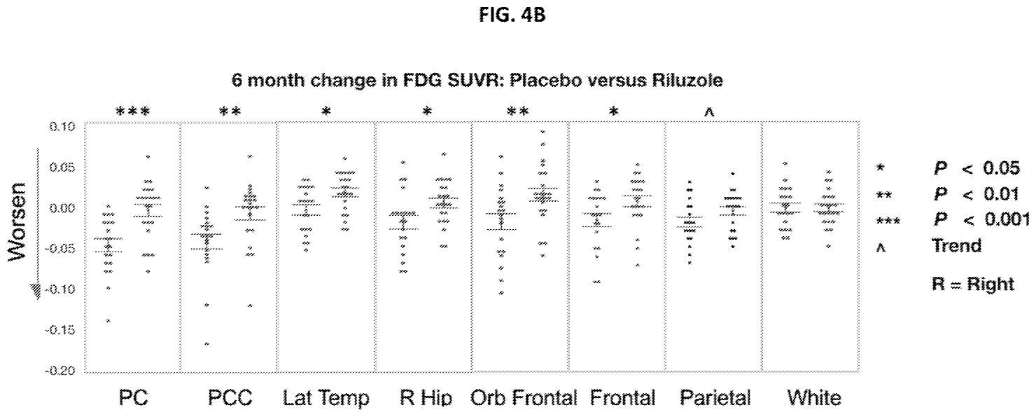

FIG. 4: (A) Region of interest boundaries shown in representative slices, coded to indicate the significance levels in comparisons between placebo and riluzole treated arms of the 6 month change in FDG SUVR; and (B) comparison between placebo and riluzole treated arms of the 6 month change in FDG SUVR for posterior cingulate (PostCing), combined PC and precuneus (PCC), lateral temporal (LatTemp), right hippocampus (Hip), orbitofrontal (OrbFrontal), Frontal, Parietal, and subcortical white matter (as a comparator, expected to remain stable). Individual values are shown with mean and standard error bars.

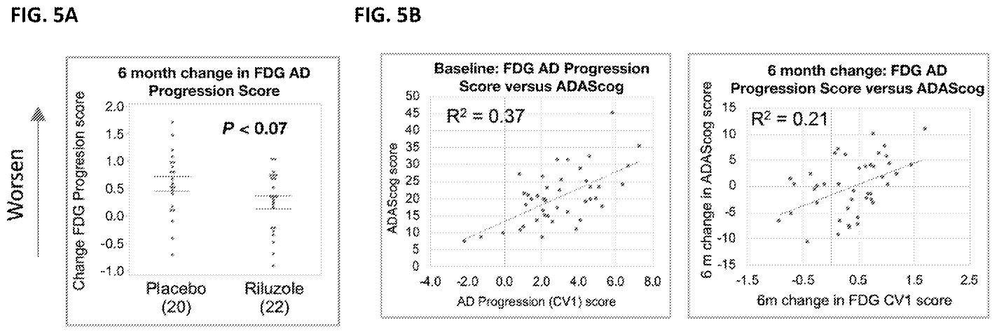

FIG. 5: (A) Comparison between placebo and riluzole treated arms of the change in FDG Progression score. (B) Correlation between AD Progression score at baseline and ADAScog score at baseline (left) and between 6 month change in AD progression score and in ADAS cog (right) and for all study participants.

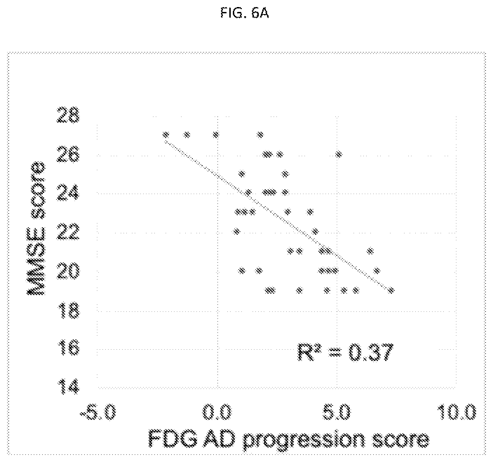

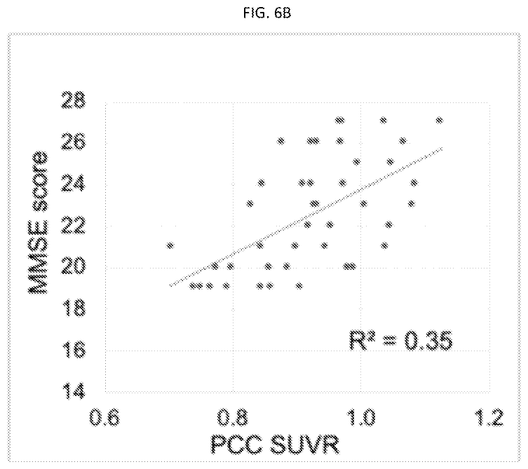

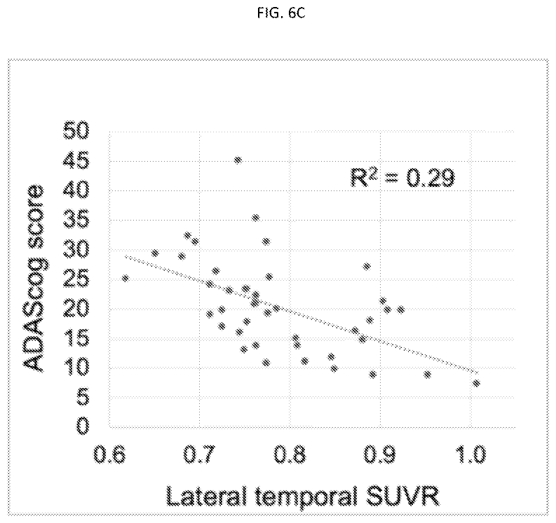

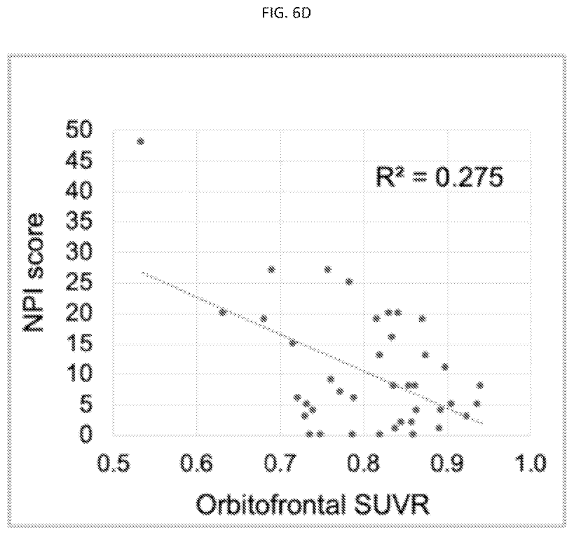

FIG. 6: Correlations at baseline between: (A) FDG AD Progression score and MMSE score; (B) posterior cingulate-precuneus (PCC) score and MMSE score; (C) lateral temporal FDG SUVR and ADAScog score; and (D) orbitofrontal FDG SUVR and Neuropsychiatric Inventory (NPI) score.

FDG PET Methods

For each FDG PET scan, a 5 mCi dose of florodeoxyglucose was administered followed by a 40 minute uptake period during which the participant was in a resting state with eyes open, without activity or audiovisual distraction. Images were acquired on a Siemens Biograph64mCT scanner as a series of 4 frames of 5 minutes each. In some of the first cases, a full dynamic scan was performed and the late timeframes were extracted for processing and analysis.

All PET images were inspected for excessive motion or other artifact. Using SPM12 (Wellcome Trust), motion correction was performed and frames averaged into a static image. Each 6 month scan was coregistered to the baseline FDG scan, which was co-registered to the participant's Tl-weighted MRI scan. The MRI scans were segmented into gray, white, and CSF tissue and spatially transformed to a template in MNI space, and the spatial transformations also applied to the PET scans.

Regions of interest (FIG. 1A[i]) adapted from the Freesurfer atlases were thresholded with a smoothed gray segment specific to each participant and the average intensities within each region of interest were measured. In addition, each image was analyzed using a previously developed AD Progression Classifier (FIG. 1A[ii]) that quantifies the degree to which a pattern of hypometabolism and preservation relative to whole brain is expressed. Increases in classifier score correspond to increased expression of a pattern of hypometabolism that corresponds to the progression of AD as validated using over 500 ADNI subjects. A reference region for calculation of Standardized Uptake Value Ratios (SUVRs) was defined based upon the voxels of preservation in the AD Progression Classifier, which are most pronounced in the paracentral region. Longitudinal changes in SUVRs using this reference were compared to SUVRs using (each separately) centrum semiovale, cerebellum, pons, and whole brain. While these regions tend to be noisier due to technical factors (cerebellum, pons), or affected by progressive hypometabolism (whole brain), or potentially affected by riluzole (cerebellum), directional concordance supported robustness of findings when the primary reference was used.

Statistical Analysis

Placebo and treatment groups were compared to identify potential baseline differences in region of interest SUVRs and in age, sex, ApoE4 dose and carrier status, and MMSE score. The 6-month change in SUVR in each region of interest was compared across groups using a one-way ANCOVA model with the change in SUVR value as the dependent variable, study arm as the categorical independent variable, and baseline SUVR value as a continuous variable covariate (JMP v15, SAS software) (Results were consistent with use of post-treatment SUVR as the independent variable). Age, gender, ApoE4 carrier status, and baseline MMSE were investigated as covariates. Assumptions including normal distribution, homogeneity of variance, and linear correlation between baseline and post-treatment SUVR were verified, and the number of covariates in a given parametric model was limited to 1-3. Non-parametric tests were applied depending upon the number of subjects per analysis group and other assumption tests. Effect sizes were calculated using Cohen's d (d). For the two participants who received their FDG PET scan 2 and 3 months after the 6-month timepoint due to restrictions arising from COVID 19, the change in value was adjusted using a linear proportional reduction (e.g. value x 6/8 or 6/9). Groups were evaluated post-hoc without these two participants. In this exploratory study, a P-value of less than 0.05 was considered significant and correction for multiple comparisons was not pre-specified. However, results using a Bonferroni correction for multiple comparisons were also reported for significant primary endpoints.

Non-prespecified FDG were evaluated in the same manner as pre-specified outcomes, without correction for multiple comparisons. The study was not statistically powered for clinical endpoints, but directional trends were examined for potential effect.

FDG PET: Baseline SUVR values and the AD Progression scores were compared between placebo and treatment groups at baseline. The 6 month change in SUVR values in each region of interest were compared across groups using an ANCOVA model (JMP v15.1), with age, gender, APOE4 carrier status, baseline SUVR, and baseline MMSE investigated as covariates. For the two participants who received their FDG PET scan 2 and 3 months after the 6 month timepoint due to COVID 19, the change in value was adjusted using a linear proportional reduction (e.g. value x 6/8 or 6/9). Groups were evaluated with and without these two participants. The AD Progression Classifier score was evaluated in the same manner.

Sample Characteristics and Demographics

A total of 94 participants were screened at the two performance sites, of which 44 did not meet inclusion/exclusion criteria. The remaining 50 participants were randomly assigned to receive riluzole (n=26) or placebo (n=24). Of these, 22 patients receiving riluzole and 20 patients receiving placebo completed the study and had both FDG PET timepoints. The diagram of FIG. 2 shows the subject disposition.

Enrolled subjects were 26 female and 16 male, age 58 to 88, and 58% apolipoprotein 4 (ApoE4) carriers (Table 1, 1 subject unavailable). There were no significant between-group differences in baseline characteristics of the patients with respect to age, sex, education, or ApoE4 in the riluzole group. Baseline neuropsychological measures were well balanced for MMSE, NPI, ADL total, CDR total in riluzole group in comparison to placebo; however, on ADAS-cog scores, riluzole group trended to be more impaired than placebo at baseline (p=0.08); Table 1).

TABLE 1
DEMOGRAPHIC AND BASELINE
CLINICAL CHARACTERISTICS

| | Placebo | | Riluzole | | |
|---|---|---|---|---|---|
| Characteristic | (n = 20) | | (n = 22) | | P-Value |
| Age (years), mean ± SD | 74.6 ± 7.7 | | 75.3 ± 5.8 | | 0.73 |
| Sex, no. (%) | | | | | 0.30 |
| Female | 14 | (70.0%) | 12 | (54.5%) | |
| Male | 6 | (30.0%) | 10 | (45.5%) | |
| Race/ethnicity, no. (%) | | | | | 1.00 |
| Black or African American | 0 | (0%) | 1 | (4.5%) | |
| Black/non-Hispanic | 1 | (5.0%) | 0 | (0%) | |
| Latino/Hispanic | 0 | (0%) | 1 | (4.5%) | |
| White/non-Hispanic | 19 | (95.0%) | 20 | (90.9%) | |
| Education (years), mean ± SD | 15.1 ± 3.1 | | 15.9 ± 3.0 | | 0.39 |
| ApoE4 carrier, no. (%) | 8 | (40.0%) | 15 | (68.2%) | 0.11 |

| | | | |
|---|---|---|---|
| Clinical scales*, mean ± SD | | | |
| ADAS-cog | 17.9 ± 7.5 | 22.5 ± 7.9 | 0.08 |
| ADL total | 68.1 ± 9.3 | 68.4 ± 9.5 | 0.91 |
| CDR-sum of boxes | 3.6 ± 1.8 | 3.8 ± 1.9 | 0.73 |
| CDR total | 0.6 ± 0.2 | 0.6 ± 0.2 | 0.59 |
| MMSE | 22.8 ± 2.9 | 22.5 ± 2.5 | 0.72 |
| NPI | 10.2 ± 11.1 | 9.6 ± 9.2 | 0.86 |
| GDS | 5.3 ± 3.7 | 5.2 ± 6.6 | 0.98 |

ADAS-cog = Alzheimer's Disease Assessment Scale, ADL = Activities of Daily Living Inventory scale, CDR = Clinical Dementia Rating scale, MMSE = Mini-Mental State Examination, NPI = Neuropsychiatric Inventory score, GDS = Geriatric Depression Scale.

Neuroimaging Outcome Measures

FDG PET

The inventors found a difference between arms in FDG PET cerebral metabolic changes over the 6 month treatment period, with less decline in multiple pre-specified brain regions in the riluzole group in comparison to placebo group. They discovered no significant or trend level differences between study arms at baseline in the regional SUVRs that were compared, or in the FDG AD Progression score. Given the trend level difference between study arms in ApoE4 dose, analyses were performed and compared with and without its inclusion as a covariate. Table 3 presents the mean, standard deviation, and significance findings for the FDG PET comparisons.

The inventors found the most robust effect of riluzole treatment in the posterior cingulate where they observed a slower decline of glucose metabolism in riluzole relative to placebo (FIG. 3A-C). Posterior cingulate (PC) glucose metabolism, a primary endpoint, was significantly preserved in riluzole-treated group in comparison to placebo over the 6 month period (effect size (d) 1.31; P<0.0002 with ApoE4 dose included as covariate, P<0.0003 without ApoE4 dose included, with the effect significant using any of several different reference regions (paracentral p<0.0002, centrum ovale p<0.008, whole brain p<0.016, cerebellar cortex p<0.03). Posterior cingulate is a hub network region and one of the areas of the brain earliest and most strongly affected in AD as demonstrated by multiple neuroimaging modalities (Minoshima et al., 1997; Mutlu et al., 2016).

PC significance readily survived Bonferroni correction for multiple comparisons. Regional cerebral glucose metabolism was more preserved in the riluzole group in comparison to placebo in several other pre-specified regions of interest including precuneus (P<0.007, d=0.84), lateral temporal (P<0.014, d=0.80), right hippocampus (P<0.025, d=0.72), and frontal cortex (P<0.035, d=0.67), and the exploratory subregions of orbitofrontal cortex (P<0.008, d=0.86) and posterior cingulate-precuneus subregion (P<0.007, d=0.88); FIG. 4. A majority of these still showed trend level significance if corrected for multiple comparisons. Age, sex, education, and ApoE4 dose were not significant contributors to treatment effect. No differences were observed in control regions such as subcortical white matter, pons, and cerebellar vermis.

When groups were stratified and analyzed separately on a post-hoc basis (using nonparametric tests due to subgroup size) by ApoE4 carrier status, age, and sex, the inventors observed less decline among the group treated with riluzole than with placebo in both ApoE4 carriers and non-carriers (P<0.004 in carriers (N=8 placebo, 15 riluzole, effect size 1.526) and P<0.09 in non-carriers (N=11 placebo, 7 riluzole, d=0.89), in both younger and older groups (P<0.002 in older group, N=13 placebo, 15 riluzole, d=1.370 and P<0.08 in younger group, N=7 placebo and 7 riluzole, d=0.96) (FIG. 3D) and in males and females (both groups p<0.02; N=14 placebo, 12 riluzole in female group and N=6 placebo, 10 riluzole in the male group). Inclusion of ApoE4 dose in the analysis by-age group increased the p value for study arm to 0.13 in the younger group, with ApoE4 dose showing a trend level influence in this age group (P<0.07) but not in the older age group (P<0.78). Inclusion of ApoE4 dose in group analyzed by sex of the subject decreased the P value for study arm to P<0.008 in the female group, with ApoE4 dose showing a trend level influence in the female group (P<0.09) but not the male group (P<0.84).

FDG PET measures have been shown to correlate with cognitive decline and predict disease progression (Alexander et al., 2002; Landau et al., 2011; Khosravi et al., 2019). The inventors found that FDG PET progression classifier scores showed a trend-level slower disease progression in the riluzole-treated group than in the placebo group (p<0.07, FIG. 5A). There was a trend level with greater difference between arms in ApoE4 carriers than noncarriers. The inventors observed a strong correlation between the FDG PET AD Progression Classifier score and ADAS-cog at baseline and in treatment change from baseline to 6 months (FIG. 5B). FDG PET AD progression scores correlated with ADAS-cog (R=0.61, p<0.00002) and changes in FDG AD progression scores correlated with changes in ADAScog (R=0.46, p<0.002) (FIG. 5B) over the 6 months of the study. The inventors observed additional correlations between FDG PET and cognitive measures as shown in FIG. 6, including relationships between baseline FDG AD Progression score and MMSE (R=0.61, p<0.00002, FIG. 6A), FDG PC SUVR and MMSE (R=0.35, p<0.00003, FIG. 6B), lateral temporal SUVR and ADAS-cog (R=0.54, p<0.0002, FIG. 6C) and orbitofrontal SUVR and NPI score (R=0.52, p<0.0004, FIG. 6D). The robust correlations observed between FDG PET brain metabolism and cognitive measures in the present dataset are in accordance with the inventors' secondary outcome measure related to neuropsychological assessment.

Adverse Events

The inventors found no statistical differences in adverse events in treatment groups, with 23 of 26 patients (88.5%) in the riluzole group and 22 of 24 (91.7%) in the placebo group having at least one adverse event during the study. Serious adverse events occurred in 2 (7.7%) in the riluzole group and 1 (4.2%) in the placebo group. The most common side effects in the riluzole group consisted of abdominal discomfort (15.4% in riluzole and none in placebo); diarrhea (15.4% in riluzole and 8.3% in placebo); dizziness (15.4% in riluzole and 4.2% in placebo); urinary frequency (11.5% in riluzole and none in placebo), nausea (7.7% in riluzole and none in placebo), cough (19.23% in riluzole and 12.5% in placebo), elevated liver enzymes (7.7% in riluzole and 4.2% in placebo) and others. (Table 2) Among the randomized patients, 4 of 26 (15.4%) in the riluzole group and 3 of 24 (12.5%) in the placebo group had an adverse event that led to removal from the trial. There were no significant differences in the frequency of participants who were discontinued from the trial due to adverse events.

TABLE 2
NUMBER (PERCENT) OF ADVERSE EVENTS

| | Riluzole | Placebo |
|---|---|---|
| EVENT | (n = 26) | (n = 24) |
| Any adverse event | 23 (88.5) | 22 (91.7) |
| Any serious adverse event | 2 (7.7) | 1 (4.2) |
| Cough | 5 (19.2) | 3 (12.5) |
| Abdominal discomfort | 4 (15.4) | 0 (0) |
| Diarrhea | 4 (15.4) | 2 (8.3) |
| Dizziness | 4 (15.4) | 1 (4.2) |
| Urinary frequency | 3 (11.5) | 0 (0) |
| Nausea | 2 (7.7) | 0 (0) |
| Back pain | 2 (7.7) | 2 (8.3) |
| Anxiety | 2 (7.7) | 4 (16.7) |
| Elevated liver enzymes | 2 (7.7) | 1 (4.2) |
| Paranoia | 1 (3.9) | 3 (12.5) |
| Rash | 1 (3.9) | 2 (8.3) |
| Fatigue | 1 (3.9) | 2 (8.3) |
| Somnolence | 0 (0) | 3 (12.5) |

FDG PET Comparisons

Table 3 presents the mean, standard deviation, and significance findings for the FDG PET comparisons.

TABLE 3
FDG PET LONGITUDINAL CHANGE OVER 6 MONTHS

| | Placebo | | Riluzole | | Comparison | |
|---|---|---|---|---|---|---|
| | | 95% | | 95% | | Effect |
| | Mean (SD) | CI Mean | Mean (SD) | CI Mean | p-value | size (d) |
| Posterior | −0.048 | (0.065, | −0.005 | (−0.021, | 0.0002 | 1.31 |
| cingulate | (0.035) | −0.031) | (0.035) | 0.011) | | |
| Precuneus | −0.032 | (−0.045, | (−0.007, | (−0.021, | 0.007 | 0.84 |
| | (0.028) | −0.018) | 0.032) | 0.008) | | |
| Temporal | −0.023 | (−0.038, | 0.002 | (−0.010, | 0.014 | 0.80 |
| | (0.033) | −0.007) | (0.029) | 0.015) | | |
| Frontal | −0.129 | (−0.159, | −0.077 | (−0.109, | 0.031 | 0.70 |
| | (0.066) | −0.098) | (0.072) | −0.045) | | |
| Parietal | −0.020 | (−0.032, | −0.005 | (−0.016, | 0.09 | 0.54 |
| | (0.027) | −0.007) | (0.024) | 0.005) | | |
| Hippocampus | −0.018 | (−0.034, | −0.002 | (−0.015, | 0.11 | 0.50 |
| | (0.034) | −0.002) | (0.029) | 0.011) | | |
| Right | −0.021 | (−0.038, | 0.002 | (−0.010, | 0.025 | 0.72 |
| Hippocampus | (0.036) | −0.004) | (0.027) | 0.014) | | |
| AD | 0.579 | (0.294, | 0.245 | (−0.003, | 0.07 | 0.57 |
| Progression | (0.607) | 0.863) | (0.558) | 0.493) | | |
| score | | | | | | |
| Post Cing- | −0.041 | (−0.061, | −0.006 | (−0.023, | 0.007 | 0.88 |
| Precuneus | (0.042) | −0.021) | (0.038) | 0.011) | | |
| Orbitofrontal | −0.019 | (−0.039, | 0.014 | (−0.002, | 0.008 | 0.86 |
| | (0.044) | 0.002) | (0.036) | 0.030) | | |

What is claimed is:

1. A method of treating Alzheimer's disease or mild cognitive impairment comprising administering to a subject in need thereof a therapeutically effective amount of riluzole.

2. The method of claim 1, wherein the therapeutically effective amount for treating Alzheimer's disease or mild cognitive impairment is a dose of about 50 mg to 300 mg per day of riluzole.

3. The method of claim 2, wherein the dose of riluzole is administered twice a day.

4. The method of claim 2, wherein a full dose of riluzole is 100 mg, which is administered as a 50 mg dose of riluzole twice per day.

5. The method of claim 2, wherein the subject manifests a reduced rate of change in the clinical manifestations of Alzheimer's Disease when compared to untreated subjects.

6. The method of claim 5, wherein methods used to measure clinical markers of Alzheimer's Disease comprise neuropsychological tests or neurocognitive tests, such as, MMSE (mini mental state exam), MOCA (Montreal Cognitive Assessment), ADAS-Cog (Alzheimer's disease Assessment Cognitive Subscale), ADL (Activities of Daily Living), NPI (Neuropsychiatry Inventory), or CDR (Clinical Dementia Rating).

7. The method of claim 2, wherein biological markers of Alzheimer's disease progression in the subject show a reduced rate of progression as compared to untreated subjects.

8. The method of claim 7, wherein the biological markers comprise one or more of brain atrophy measurements, hippocampal and cortical volume measurements, neuroimaging biomarkers, fluorodeoxyglucose (FDG)-positron emission tomography (PET), tau-PET, amyloid PET, blood or cerebrospinal fluid (CSF) biomarkers of neurofilament, beta-amyloid, phospholyrated tau.

9. The method of claim 8, wherein the biological markers comprise changes in cerebral glucose metabolism in at least one of the: posterior cingulate, hippocampus, precuneus, medial temporal, lateral temporal, inferior parietal, or frontal cortex.

10. A method of treating mild to moderate Alzheimer's disease or mild cognitive impairment comprising administering to a subject in need thereof a therapeutically effective amount of riluzole.

11. The method of claim 10, wherein the therapeutically effective amount for treating said mild to moderate Alzheimer's disease or mild cognitive impairment is a dose of about 50 mg to 300 mg per day of riluzole.

12. The method of claim 11, wherein the dose of riluzole is administered at least twice a day.

13. The method of claim 11, wherein a full dose of riluzole is 100 mg, which is administered as a 50 mg dose of riluzole at least twice per day.

14. The method of claim 11, wherein the subject has mild Alzheimer's Disease.

15. The method of claim 14, wherein the therapeutically effective amount for treating mild Alzheimer's disease is a dose of about 50 mg to 300 mg per day of riluzole.

16. The method of claim 15, wherein the dose of riluzole is administered twice a day.

17. The method of claim 16, wherein a full dose of riluzole is 100 mg, which is administered as a 50 mg dose of riluzole at least twice per day.

18. The method of claim 10, wherein the therapeutically effective amount for treating mild cognitive impairment is a dose of about 50 mg to 300 mg per day of riluzole.

19. The method of claim 18, wherein the dose of riluzole is administered twice a day.

20. The method of claim 19, wherein a full dose of riluzole is 100 mg, which is administered as a 50 mg dose of riluzole twice per day.
